SEQUENCE LISTING (5 sequences)

SEQ ID NO: 1 - RNA, 12 nt, Artificial Sequence
  Synthetic_Essential part of crRNA
guuuuagagcua

SEQ ID NO: 2 - RNA, 10 nt, Artificial Sequence
  Synthetic_3′-terminal part of crRNA
ugcuguuuug

SEQ ID NO: 3 - RNA, 60 nt, Artificial Sequence
  Synthetic_Essential part of tracrRNA
uagcaaguuaaaauaaggcuaguccguuaucaacuugaaaaaguggcaccgagucggugc

SEQ ID NO: 4 - protein, 1368 aa, Artificial Sequence [sequence omitted]

SEQ ID NO: 5 - DNA, 20 bp, Artificial Sequence
  Synthetic_Target site of human AAVS1
gtcaccaatcctgtccctag

(19) United States
(12) Patent Application Publication
(10) Pub. No.: US 2020/0368284 A1
(43) Pub. Date: Nov. 26, 2020

(54) ANGIOPOIETIN-1- OR VEGF-SECRETING STEM CELL AND PHARMACEUTICAL COMPOSITION FOR PREVENTION OR TREATMENT OF CARDIOVASCULAR DISEASE, COMPRISING SAME

(71) Applicant: NSAGE CORP., Incheon (KR)

(72) Inventors: Bonghee LEE, Jeju-si (KR); Delger Bayarsaikhan, Ulaanbaatar (MN); Jaeseok LEE, Incheon (KR)

(21) Appl. No.: 16/623,868

(22) PCT Filed: Jun. 19, 2018
(86) PCT No.: PCT/KR2018/006920

§ 371 Date: Dec. 18, 2019

(30) Foreign Application Priority Data
Jun. 20, 2017 (KR) 10-2017-0078197

(51) Int. Cl. A61K 35/28; A61K 38/18; A61P 9/10

(52) U.S. Cl. CPC A61K 35/28; A61P 9/10; A61K 38/1891; A61K 38/1866

(57) ABSTRACT

Disclosed are angiopoietin-1 (Ang-1)- or VEGF-secreting stem cells promotive of vascular formation, a generation method therefor, and a use thereof in preventing or treating cardiovascular disease.

### TECHNICAL FIELD

Provided are stem cells secreting angiopoietin-1 (Ang-1) or VEGF, which promote vascular formation, a preparation method therefor, and a use thereof in preventing or treating a cardiovascular disease.

### BACKGROUND ART

Active research into cell therapy products are ongoing in the field of pharmacology. Representative of cell therapy products are myocardial stem cells. Mesenchymal stem cells, hematopoietic stem cells, endothelial precursor cells, myoblasts, and adipocyte-derived stem cells are arising as stem cells differentiating into myocardial stem cells. As such, the differentiated myocardial stem cells have been added with increasing usefulness as cell therapy products for cardiovascular disease, and active research into the development techniques therefor is ongoing.

However, stem cells produced by simple isolation culturing methods in current use are poor in therapeutic efficacy. Therefore, there is a need for the development of next-generation stem cells that exhibit more potent therapeutic functions at high efficacy.

### DETAILED DESCRIPTION OF THE INVENTION

### Technical Problem

An embodiment provides an Ang-1-secreting stem cell, which secretes Ang-1.

Another embodiment provides a VEGF-secreting cell, which secretes VEGF.

Another embodiment provides an Ang-1- and VEGF-secreting stem cell comprising an Ang-1-secreting stem cell and a VEGF-secreting stem cell.

The stem cells may be human-derived stem cells. The Ang-1- and/or VEGF-secreting stem cell may have an Ang-1 gene and/or a VEGF gene inserted into the genome thereof, for example, into a safe harbor, such as AAVS1, in the genome thereof. The stem cell may be a mesenchymal stem cell, for example, an umbilical cord-derived mesenchymal stem cell.

Another embodiment provides a pharmaceutical composition for vascular formation or for promoting vascular formation, the composition comprising at least one selected from the group of an Ang-1-secreting stem cell and a VEGF-secreting stem cell, or a culture thereof.

Another embodiment provides a method of vascular formation or a method of promoting vascular formation comprising a step of administering at least one selected from the group consisting of an Ang-1-secreting stem cell and a VEGF-secreting stem cell, or a culture thereof in a pharmaceutically effective amount, to a subject in need of vascular formation or promoting vascular formation.

Another embodiment provides a pharmaceutical composition for inhibition of ischemic cell death, the composition comprising at least one selected from the group of an Ang-1-secreting stem cell and a VEGF-secreting stem cell, or a culture thereof.

Another embodiment provides a method for inhibition of ischemic cell death, the method comprising a step of administering to a subject in need thereof at least one selected from the group of an Ang-1-secreting stem cell and a VEGF-secreting stem cell, or a culture thereof in a pharmaceutically effective amount.

Another embodiment provides a pharmaceutical composition comprising at least one selected from the group of an Ang-1-secreting stem cell and a VEGF-secreting stem cell, or a culture thereof as an effective ingredient for prevention or treatment of cardiovascular disease.

Another embodiment provides a method for prevention or treatment of cardiovascular disease, the method comprising a step of administering to a subject in need thereof at least one selected from the group of an Ang-1-secreting stem cell and a VEGF-secreting stem cell, or a culture thereof in a pharmaceutically effective amount.

The cardiovascular disease is caused by cardiovascular abnormality and may be selected from all ischemic cardiovascular diseases, for example, may be one selected from the group, but not limited to, stroke, myocardial infarction, angina pectoris, lower limb ischemia, hypertension, and arrhythmia.

Another embodiment provides a method for preparation of a stem cell secreting either or both of Ang-1 and VEGF, the method comprising a step of introducing either or both of an Ang-1 gene and a VEGF gene into the genome of a stem cell. The step of introducing an Ang-1 gene and/or a VEGF gene into the genome of a stem cell may be carried out by an endonuclease (or a nucleic acid molecule coding therefor) and a guide RNA (or a nucleic acid molecule coding therefor). The endonuclease may be an RNA-guided endonuclease (RGEN).

The endonuclease and the guide RNA may be used (i.e., administered) in the form of:

(1) a ribonucleoprotein in which an endonuclease protein is associated with guide RNA to form a complex; or

(2) a mixture of (a) an endonuclease protein, a nucleic acid molecule coding therefor, or a recombinant vector carrying the nucleic acid molecule and (b) a guide RNA, a nucleic acid molecule coding for the guide RNA, or a recombinant vector carrying the nucleic acid molecule.

Another embodiment provides an Ang-1- and VEGF-secreting stem cell prepared by the preparation method.

Another embodiment provides an endonuclease (or nucleic acid molecular coding therefor)/guide RNA (or nucleic acid molecule therefor) complex, for example, CRISPR/Cas9 RNP, for use in constructing an Ang-1- and VEGF-secreting stem cell.

### Technical Solution

Intensive and thorough research, conducted by the present inventors, into vasculature regeneration in ischemic disease, resulted in the finding that stem cells generated to secrete angiopoietin-1 (Ang-1) and vascular endothelial growth factor (VEGF) in a myocardial infarction model or a lower limb ischemia model can be used to prevent or treat ischemic cardiovascular disease.

An embodiment provides an Ang-1-secreting stem cell, which secretes Ang-1.

Another embodiment provides a VEGF-secreting cell, which secretes VEGF.

Another embodiment provides an Ang-1- and VEGF-secreting stem cell comprising an Ang-1-secreting stem cell and a VEGF-secreting stem cell.

The stem cells may be human-derived stem cells. The Ang-1- and/or VEGF-secreting stem cell may have an Ang-1 gene and/or a VEGF gene inserted into the genome thereof, for example, into a safe harbor, such as AAVS1, in the genome thereof. The stem cell may be a mesenchymal stem cell, for example, an umbilical cord-derived mesenchymal stem cell.

Another embodiment provides a pharmaceutical composition for vascular formation or for promoting vascular formation, the composition comprising at least one selected from the group of an Ang-1-secreting stem cell and a VEGF-secreting stem cell, or a culture thereof.

Another embodiment provides a method of vascular formation or a method of promoting vascular formation comprising a step of administering at least one selected from the group consisting of an Ang-1-secreting stem cell and a VEGF-secreting stem cell, or a culture thereof in a pharmaceutically effective amount, to a subject in need of vascular formation or promoting vascular formation.

Another embodiment provides a pharmaceutical composition for inhibition of ischemic cell death, the composition comprising at least one selected from the group of an Ang-1-secreting stem cell and a VEGF-secreting stem cell, or a culture thereof.

Another embodiment provides a method for inhibition of ischemic cell death, the method comprising a step of administering to a subject in need thereof at least one selected from the group of an Ang-1-secreting stem cell and a VEGF-secreting stem cell, or a culture thereof in a pharmaceutically effective amount.

The term “ischemic cell death”, as used herein, refers to the cell death of cardiomyocyte or myocytes attributed to the interruption or deficiency of blood supply through vessels, which is caused by cardiovascular diseases such as stroke, myocardial infarction, angina pectoris, lower limb ischemia, hypertension, arrhythmia, and so forth, or to immunological causes. The Ang-1- and/or VEGF-secreting stem cells provided in the present disclosure can effectively inhibit the induction of such ischemic cell death whereby ischemic cardiovascular disease can be prevented and/or treated.

Another embodiment provides a pharmaceutical composition comprising at least one selected from the group of an Ang-1-secreting stem cell and a VEGF-secreting stem cell, or a culture thereof as an effective ingredient for prevention or treatment of cardiovascular disease.

Another embodiment provides a method for prevention or treatment of cardiovascular disease, the method comprising a step of administering to a subject in need thereof at least one selected from the group of an Ang-1-secreting stem cell and a VEGF-secreting stem cell, or a culture thereof in a pharmaceutically effective amount.

The cardiovascular disease is caused by cardiovascular abnormality and may be selected from all ischemic cardiovascular diseases, for example, may be one selected from the group consisting of, but not limited to, stroke, myocardial infarction, angina pectoris, lower limb ischemia, hypertension, and arrhythmia.

Another embodiment provides a method for preparation of a stem cell secreting either or both of Ang-1 and VEGF, the method comprising a step of introducing either or both of an Ang-1 gene and a VEGF gene into the genome of a stem cell. The step of introducing an Ang-1 gene and/or a VEGF gene into the genome of a stem cell may be carried out by an endonuclease (or a nucleic acid molecule coding therefor) and a guide RNA (or a nucleic acid molecule coding therefor). The endonuclease may be an RNA-guided endonuclease (RGEN).

The endonuclease and the guide RNA may be used (i.e., administered) in the form of:
(1) a ribonucleoprotein in which an endonuclease protein is associated with guide RNA to form a complex; or
(2) a mixture of (a) an endonuclease protein, a nucleic acid molecule coding therefor, or a recombinant vector carrying the nucleic acid molecule and (b) a guide RNA, a nucleic acid molecule coding for the guide RNA, or a recombinant vector carrying the nucleic acid molecule.

Another embodiment provides an Ang-1- and VEGF-secreting stem cell prepared by the preparation method.

Another embodiment provides an endonuclease (or nucleic acid molecular coding therefor)/guide RNA (or nucleic acid molecule therefor) complex, for example, CRISPR/Cas9 RNP, for use in constructing an Ang-1- and VEGF-secreting stem cell.

As used herein, the term “Ang-1-secreting stem cell” means a stem cell which has an Ang-1 gene introduced thereinto and secretes Ang-1, the term “VEGF-secreting stem cell” means a stem cell which has a VEGF gene introduced thereinto and secretes VEGF, and the term “Ang-1 and VEGF-secreting stem cell” means a mixture of the Ang-1-secreting stem cell and the VEGF-secreting stem cell or a stem cell which has both an Ang-1 gene and a VEGF gene introduced thereinto and secretes both Ang-1 and VEGF. Herein, the “Ang-1-secreting stem cell”, “VEGF-secreting stem cell”, and “Ang-1- and VEGF-secreting stem cell” may be referred to as “Ang-1- and/or VEGF-secreting stem cell”.

Herein, the Ang-1- and VEGF-secreting stem cell has an vascular formation promoting effect (increase in vascularization rate and/or angiogenic or vasculogenic factor production) and/or an ischemic cell death inhibiting effect, exhibiting the prevention, symptom alleviation or reduction, and treatment of cardiovascular disease. The cardiovascular disease that can be treated with the Ang-1- and VEGF-secreting stem cell may include all ischemic cardiovascular diseases, for example, may be at least one selected from the group consisting of, but not limited to, myocardial infarction, angina pectoris, lower limb ischemia, and stroke.

The subject may be selected from mammals including primates such as humans, apes, and the like and rodents such as rats, mice, and the like, which suffer from an ischemic cell death symptom and/or cardiovascular disease, cells (cardiomyocytes or cardiovascular cells) or tissues (cardiac tissues) isolated from the mammals, or cultures thereof. By way of example, selection may be made of a human suffering from an ischemic cell death symptom or cardiovascular disease, cardiomyocytes, cardiovascular cells, cardiac tissues isolated therefrom, or a culture of the cells or tissues.

The Ang-1- and/or VEGF-secreting stem cell provided as an effective ingredient in the disclosure or a pharmaceutical composition comprising the same may be administered to the subject via various routes including oral and parenteral routes, e.g., subcutaneously, intradermaliy, intratumorally, intranodally, intramedullary, intramuscularly, intravenously, intralymphatically, intraperitoneally, or intralesionally. For example, the Ang-1- and/or VEGF-secreting stem cell or the composition containing the same may be administered in any convenient way, such as injection, transfusion, implantation, or transplantation into a lesion site (e.g., heart (cardiomyocytes, cardiac vessels, etc.)) of a subject, or via vessel routes (vein or artery), without any limitation thereto.

The pharmaceutical compositions provided herein may be formulated according to conventional methods into oral dosage forms such as powders, granules, tablets, capsules, suspensions, emulsions, syrups, aerosols, or parenteral dosage forms such as suspensions, emulsions, lyophilized agent, external preparations, suppositories, sterile injectable solutions, implant preparations, and the like.

The amount of the stem cells or the pharmaceutical composition of the present disclosure may vary depending on the age, sex, and weight of the subject to be treated, and above all, the condition of the subject to be treated, the specific category or type of disease to be treated, the route of administration, the nature of the therapeutic agent used, and the sensitivity to specific therapeutic agents, and may be prescribed in consideration thereof. For example, the stem cells may be administered to a subject at a dose of 1×10³-1×10⁹ cells, e.g., 1×10⁴-1×10⁹ cells, 1×10⁴-1×10⁸ cells, 1×10⁵-1×10⁷ cells, or 1×10⁵-1×10⁶ cells per kg of body weight, but is not limited thereto.

The angiopoietin-1 (Ang-1), which is a protein with a critical role in vascular development, may be at least one selected from mammalian Ang-1's including human Ang-1 (gene (mRNA): GenBank Accession No. NM_001146.4). The vascular endothelial growth factor (VEGF), which is an important protein involved in vasculogenesis and angiogenesis, may be at least one selected from mammalian VEGFs including human VEGF (gene (mRNA): GenBank Accession No. NM_001171623.1).

In the present disclosure, the stem cells may be derived from mammals, e.g., humans. As used herein, the term “stem cell” is intended to encompass all embryonic stem cells, adult stem cells, and progenitor cells. For example, the stem cells may be at least one selected from the group consisting of embryonic stem cells, adult stem cells, and progenitor cells. The stem cells may be homologous and/or autologous.

Embryonic stem cells are stem cells derived from an embryo and able to differentiate into cells of any tissue.

Progenitor cells have an ability to differentiate into a specific type of cells, but are already more specific than stem cells and are pushed to differentiate into their target cells. Unlike stem cells, progenitor cells undergo limited divisions. The progenitor cells may be derived from mesenchymal stem cells, but are not limited thereto. In the disclosure, progenitor cells fall within the scope of stem cells and unless otherwise stated, “stem cells” are construed to include progenitor cells.

Adult stem cells, which are stem cells derived from the umbilical cord, umbilical cord blood or adult bone marrow, blood, nerves, etc., refer to primitive cells immediately before differentiation into cells of concrete organs. The adult stem cells are at least one selected from the group consisting of hematopoietic stem cells, mesenchymal stem cells, neural stem cells, and the like. The adult stem cells may be derived from mammals, for example, humans. Adult stem cells are difficult to proliferate and are prone to differentiation. Instead, adult stem cells can be used not only to reproduce various organs required by actual medicine, but also to differentiate according to the characteristics of individual organs after transplantation thereto. Hence, adult stem cells can be advantageously applied to the treatment of incurable diseases.

In one embodiment, the stem cells may be mesenchymal stem cells (MSC). The term “mesenchymal stem cells”, also called mesenchymal stromal cells (MSC), means multipotent stromal cells that can differentiate into various types of cells, such as osteoblasts, chondrocytes, myocytes, adipocytes, and the like. Mesenchymal stem cells may be selected from pluripotent cells derived from non-marrow tissues such as placenta, umbilical cord blood, umbilical cord, adipose tissues, adult muscles, corneal stroma, and dental pulp from deciduous teeth. In one embodiment, the mesenchymal stem cells may be umbilical mesenchymal stem cells derived from mammals, e.g., humans.

The gene insertion may refer to the incorporation of an Ang-1 gene and/or a VEGF gene into the genome of a stem cell, for example, into a safe harbor gene site, such as AAVS1, in the genome of a stem cell. A safe harbor gene site is a genomic location where DNA may be damaged (cleaved, and/or deletion, substitution, or insertion of nucleotide(s)) without disrupting cell injury and may include, but is not limited to, AAVS1 (adeno-associated virus integration site; e.g., AAVS1 in human chromosome 19 (19q 13)).

Insertion (introduction) of the Ang-1 gene and/or the VEGF gene into a stem cell genome may be achieved using any genetic manipulation technique that is typically used to introduce a gene into a genome in an animal cell. In one embodiment, the genetic manipulation technique may employ an endonuclease. The endonuclease may target such a safe harbor gene site as is described above.

The endonuclease serves to cleave a specific site on a specific gene in a stem cell genome and to insert a foreign gene (i.e., Ang-1 gene and VEGF gene) thereinto.

As used herein, the term “endonuclease”, which is also called programmable nuclease, is intended to encompass all types of endonucleases that recognize and cleave (single-strand break or double-strand break) specific sites on target genomic DNA. The endonuclease may be an enzyme isolated from a microbe or a non-naturally occurring enzyme obtained in a recombinant or synthetic manner. The target-specific nuclease may further include an element that is typically used for intracellular delivery in eukaryotic cells (e.g., nuclear localization signal; NLS), but is not limited thereto. The target specific nuclease may be used in the form of a purified protein, a DNA encoding the same, or a recombinant vector carrying the DNA.

The endonuclease may be at least one selected from the group consisting of meganuclease, zinc finger (Fokl protein) nuclease, CRISPR/Cas9 (Cas9 protein), CRISPR-Cpf1 (Cpf1 protein), and TALE-nuclease. In one embodiment, the endonuclease may be a Cas9 protein or a Cpf1 protein.

For example, the endonuclease may be at least one selected from the group consisting of:
transcription activator-like effector nuclease (TALEN) in which a transcription activator-like (TAL) effector DNA-binding domain, derived from a gene responsible for plant infection, for recognizing a specific target sequence, is fused to a DNA cleavage domain;
zinc-finger nuclease (ZFN);
meganuclease;
RNA-guided engineered nuclease (RGEN), which is derived from the microbial immune system CRISPR, such as Cas proteins (e.g., Cas9, etc.), Cpf1, and the like; and
Ago homolog (DNA-guided endonuclease), but is not limited thereto.

The target-specific endonuclease recognizes specific base sequences in the genome of animal and plant cells (i.e., eukaryotic cells), including human cells, to cause double strand breaks (DSBs). The double strand breaks create a blunt end or a cohesive end by cleaving the double strands of DNA. DSBs are efficiently repaired by homologous recombination or non-homologous end-joining (NHEJ) mechanisms within the cell, which allows researchers to introduce desired mutations into on-target sites during this process.

The target-specific nuclease recognizes specific base sequences in the genome of animal and plant cells (i.e., eukaryotic cells), including human cells, to cause double strand breaks (DSBs). The double strand breaks create a blunt end or a cohesive end by cleaving the double strands of DNA. DSBs are efficiently repaired by homologous recombination or non-homologous end-joining (NHEJ) mechanisms within the cell, which allows researchers to introduce desired mutations into on-target sites during this process.

The meganuclease may be included within, but is not limited to, a scope of naturally occurring meganucleases. The naturally occurring meganucleases recognize 15-40 base pair-long sites to be cleaved and are commonly classified into the following families: LAGLIDADG family, GIY-YIG family, His-Cyst box family, and HNH family. Exemplary meganucleases include I-SceI, I-CeuI, PI-PspI, PI-SceI, I-SceIV, I-CsmI, I-PanI, I-SceII, I-PpoI, I-SceIII, I-CreI, I-TevI, I-TevII, and I-TevIII.

DNA-binding domains from naturally occurring meganucleases, primarily from the LAGLIDADG family, have been used to promote site-specific genome modification in plants, yeasts, Drosophila, mammalian cells, and mice, but this approach has been limited to the modification of either homologous genes that conserve the meganuclease recognition sequence (Monet et al. (1999), Biochem. Biophysics. Res. Common. 255: 88-93) or pre-engineered genomes into which a recognition sequence has been introduced. Accordingly, attempts have been made to engineer meganucleases to exhibit novel binding specificity at medically or biotechnologically relevant sites. In addition, naturally occurring or engineered DNA-binding domains from meganucleases have been operably linked to a cleavage domain from a heterologous nuclease (e.g., Fokl).

The ZFN comprises a zinc finger protein engineered to bind to a target site in a gene of interest and cleavage domain or a cleavage half-domain. The ZFN may be an artificial restriction enzyme comprising a zinc-finger DNA binding domain and a DNA cleavage domain. Here, the zinc-finger DNA binding domain may be engineered to bind to a sequence of interest. For example, reference may be made to Beerli et al. (2002) Nature Biotechnol. 20:135-141; Pabo et al. (2001) Ann. Rev. Biochem. 70:313-340; Isalan et al, (2001) Nature Biotechnol. 19: 656-660; Segal et al. (2001) Curr. Opin. Biotechnol. 12:632-637; and Choo et al. (2000) Curr. Opin. Struct. Biol. 10:411-416. Compared to a naturally occurring zinc finger protein, an engineered zinc finger binding domain can have a novel binding specificity. Engineering methods include, but are not limited to, rational design and various types of selection. Rational design includes, for example, using databases comprising triplet (or quadruplet) nucleotide sequences and individual zinc finger amino acid sequences, in which each triplet or quadruplet nucleotide sequence is associated with one or more amino acid sequences of zinc fingers which bind the particular triplet or quadruplet sequence.

Selection of target sites, and design and construction of fusion proteins (and polynucleotides encoding the same) are known to those skilled in the art and described in detail in U.S. Pat. Nos. 2005/0064474 A and 2006/0188987 A, incorporated by reference in their entireties herein. In addition, as disclosed in these and other references, zinc finger domains and/or multi-fingered zinc finger proteins may be linked together using any suitable linker sequences, including, for example, linkers of 5 or more amino acids in length. Reference may be made to U.S. Pat. Nos. 6,479,626; 6,903,185; and 7,153,949 for exemplary linker sequences of? 6 or more amino acids in length. The proteins described herein may include any combination of suitable linkers between the individual zinc fingers of the protein.

Nucleases such as ZFNs also comprise a nuclease active site (cleavage domain, cleavage half-domain). As noted above, the cleavage domain may be heterologous to the DNA-binding domain, for example, such as a zinc finger DNA-binding domain and a cleavage domain from a different nuclease. Heterologous cleavage domains can be obtained from any endonuclease or exonuclease. Exemplary endonucleases from which a cleavage domain can be derived include, but are not limited to, restriction endonucleases and meganucleases.

Similarly, a cleavage half-domain can be derived from any nuclease or portion thereof, as set forth above, which requires dimerization for cleavage activity. In general, two fusion proteins are required for cleavage if the fusion proteins comprise cleavage half-domains. Alternatively, a single protein comprising two cleavage half-domains can be used. The two cleavage half-domains can be derived from the same endonuclease (or functional fragments thereof), or each cleavage half-domain can be derived from a different endonuclease (or functional fragments thereof). In addition, the target sites for the two fusion proteins are preferably disposed, with respect to each other, such that binding of the two fusion proteins to their respective target sites places the cleavage half-domains in a spatial orientation to each other that allows the cleavage half-domains to form a functional cleavage domain, e.g., by dimerizing. Thus, in an embodiment, the near edges of the target sites are separated by 3-8 nucleotides or by 14-18 nucleotides. However, any integral number of nucleotides or nucleotide pairs can intervene between two target sites (e.g., from 2 to 50 nucleotide pairs or more). Generally, the site of cleavage lies between the target sites.

Restriction endonucleases (restriction enzymes) are present in many species and are capable of binding to DNA (at a recognition site) in a sequence-specific manner and cleaving DNA at or near the site of binding. Certain restriction enzymes (e.g., Type IIS) cleave DNA at sites removed from the recognition site and have separable binding and cleavage domains. For example, the Type IIS enzyme Fokl catalyzes double-stranded cleavage of DNA, at 9 nucleotides from its recognition site on one strand and 13 nucleotides from its recognition site on the other. Thus, in one embodiment, fusion proteins comprise the cleavage domain (or cleavage half-domain) from at least one Type IIS restriction enzyme and one or more zinc finger binding domains (which may or may not be engineered).

As used herein, the term “TALEN” refers to a nuclease capable of recognizing and cleaving a target region of DNA. TALEN is a fusion protein comprising a TALE domain and a nucleotide cleavage domain. In the present disclosure, the terms “TAL effector nuclease” and “TALEN” are interchangeably used. TAL effectors are known as proteins that are secreted by Xanthomonas bacteria via their type III secretion system when they infect a variety of plant species. The protein may be bound to a promoter sequence in a host plant to activate the expression of a plant gene that aids bacterial infection. The protein recognizes plant DNA sequences through a central repetitive domain consisting of various numbers of 34 or fewer amino acid repeats. Accordingly, TALE is considered to be a novel platform for tools in genome engineering. However, in order to construct a functional TALEN with genomic-editing activity, a few key parameters that have remained unknown thus far should be defined as follows: i) the minimum DNA-binding domain of TALE, ii) the length of the spacer between the two half-sites constituting one target region, and iii) the linker or fusion junction that links the Fokl nuclease domain to dTALE.

The TALE domain of the present disclosure refers to a protein domain that binds nucleotides in a sequence-specific manner via one or more TALE-repeat modules. The TALE domain includes, but is not limited to, at least one TALE-repeat module, and more specifically, 1 to 30 TALE-repeat modules. In the present disclosure, the terms “TAL effector domain” and “TALE domain” are interchangeable. The TALE domain may include half of the TALE-repeat module. As concerns the TALEN, reference may be made to Patent Publication No. WO/2012/093833 or U.S. Patent No. 2013-0217131 A of which the entire contents are incorporated by reference in their entireties herein.

In one embodiment, insertion (or introduction) of the Ang-1- and/or VEGF-encoding gene into a stem cell genome may be achieved using a target-specific nuclease (RGEN derived from CRISPR). The endonuclease may comprise:
(1) an RNA-guided nuclease (or a DNA coding therefor or a recombinant vector carrying the coding DNA), and
(2) a guide RNA capable of hybridizing with (or having a complementary nucleotide sequence to) a target site (e.g., a region of 15 to 30, 17 to 23, or 18 to 22 consecutive nucleotides in a safe harbor gene such as AAVS1) in a target gene (e.g., a safe harbor site such as AAVS1), or a DNA coding therefor (or a recombinant vector carrying the coding DNA).

The endonuclease may be at least one selected from all nucleases that can recognize specific sequences of target genes and have nucleotide cleavage activity to incur indel (insertion and/or deletion) in the target genes.

In one embodiment, the endonuclease may be at least one selected from the group consisting of nucleases included in the type II and/or type V CRISPR system, such as Cas proteins (e.g., Cas9 protein (CRISPR (clustered regularly interspaced short palindromic repeats) associated protein 9)), Cpf1 protein (CRISPR from Prevotella and Francisella 1), etc. In this regard, the target-specific nuclease further comprises a target DNA-specific guide RNA for guiding to a target site on a genomic DNA. The guide RNA may be an RNA transcribed in vitro, for example, RNA transcribed from double-stranded oligonucleotides or a plasmid template, but is not limited thereto. The target-specific nuclease may act in a ribonucleoprotein (RNP) form in which the nuclease is associated with guide RNA to form a ribonucleic acid-protein complex (RNA-Guided Engineered Nuclease), in vitro or after transfer to a body (cell).

The Cas protein, which is a main protein component in the CRISPR/Cas system, accounts for activated endonuclease or nickase activity.

The Cas protein or gene information may be obtained from a well-known database such as GenBank at the NCBI (National Center for Biotechnology Information). By way of example, the Cas protein may be at least one selected from the group consisting of:
a Cas protein derived from Streptococcus sp., e.g., Streptococcus pyogenes, for example, Cas9 protein (i.e., SwissProt Accession number Q99ZW2 (NP_269215.1));
a Cas protein derived from Campylobacter sp., e.g., Campylobacter jejuni, for example, Cas9 protein;
a Cas protein derived from Streptococcus sp., e.g., Streptococcus thermophiles or Streptococcus aureus, for example, Cas9 protein;
a Cas protein derived from Neisseria meningitidis, for example, Cas9 protein;
a Cas protein derived from Pasteurella sp., e.g., Pasteurella multocida, for example, Cas9 protein; and
a Cas protein derived from Francisella sp., e.g., Francisella novicida, for example, Cas9 protein, but is not limited thereto.

When the cleavage at a specific site of a gene is induced by Cas9 protein, the gene cleavage may be the cleavage at a nucleotide, e.g., single-strand or double-strand break, 3 bp ahead of the PAM sequence in consecutive 17 bp- to 30 bp-long nucleotide sequence region located adjacent to the 5′ end of the PAM on each gene, characteristic to the Cas9 protein according to the microorganisms of origin.

According to one embodiment, in a case where the Cas9 protein is derived from Streptococcus pyogenes, the PAM sequence may be 5′-NGG-3′ (N is A, T, G, or C) and the nucleotide sequence site to be cleaved (target site) may be a consecutive 17 bp- to 30 bp-long or 17 bp- to 23 bp-long, for example, 20 bp-long nucleotide sequence located adjacent to the 5′- and/or 3′-end of the 5′-NGG-3′ sequence in a target gene.

According to another embodiment, in a case where the Cas9 protein is derived from Campylobacter jejuni, the PAM sequence may be 5′-NNNNRYAC-3′ (N's are each independently A, T, C or G, R is A or G, and Y is C or T) and the nucleotide sequence site to be cleaved (target site) may be a consecutive 17 bp- to 23 bp-long, for example, 22 bp- to 23 bp-long nucleotide sequence located adjacent to the 5′- and/or 3′-end of the NNNNRYAC-3′ sequence in a target gene.

According to another embodiment, in a case where the Cas9 protein is derived from Streptococcus thermophiles, the PAM sequence may be 5′-NNAGAAW-3′ (N's are each independently A, T, C, or G, and W is A or T) and the nucleotide sequence site to be cleaved (target site) may be a consecutive 17 bp- to 23 bp-long, for example, 21 bp- to 23 bp-long nucleotide sequence located adjacent to the 5′- and/or 3′-end of the NNAGAAW-3′ sequence in a target gene.

According to another embodiment, in a case where the Cas9 protein is derived from Neisseria meningitidis, the PAM sequence may be 5′-NNNNGATT-3′(N's are each independently A, T, C or G) and the nucleotide sequence site to be cleaved (target site) may be a consecutive 17 bp- to 23 bp-long, for example, 21 bp- to 23 bp-long nucleotide sequence located adjacent to the 5′- and/or 3′-end of the 5′-NNNNGATT-3′ sequence in a target gene.

According to another embodiment, in a case where the Cas9 protein is derived from Streptocuccus aureus, the PAM sequence may be 5′-NNGRR(T)-3′ (N's are each independently A, T, C or G, R is A or G, and (T) means an optional sequence included therein) and the nucleotide sequence site to be cleaved (target site) may be a consecutive 17 bp- to 23 bp-long, for example, 21 bp- to 23 bp-long nucleotide sequence located adjacent to the 5′- and/or 3′-end of the 5′-NNGRR(T)-3′ sequence in a target gene.

The Cpf1 protein, which is an endonuclease in a new CRISPR system distinguished from the CRISPR/Cas system, is small in size relative to Cas9, requires no tracrRNA, and can act with the guidance of single guide RNA. In addition, the Cpf1 protein recognizes a thymine-rich PAM (protospacer-adjacent motif) sequence and cleaves DNA double strands to form a cohesive end (cohesive double-strand break).

By way of example, the Cpf1 protein may be derived from Candidatus spp., Lachnospira spp., Butyrivibrio spp., Peregrinibacteria, Acidominococcus spp., Porphyromonas spp., Prevotella spp., Francisella spp., Candidatus Methanoplasma, or Eubacterium spp., e.g., from Parcubacteria bacterium (GWC2011_GWC2_44_17), Lachnospiraceae bacterium (MC2017), Butyrivibrio proteoclasiicus, Peregrinibacteria bacterium (GW2011_GWA_33_10), Acidaminococcus sp. (BV3L6), Porphyromonas macacae, Lachnospiraceae bacterium (ND2006), Porphyromonas crevioricanis, Prevotella disiens, Moraxella bovoculi (237), Smiihella sp. (SC_KO8D17), Leptospira inadai, Lachnospiraceae bacterium (MA2020), Francisella novicida (U112), Candidatus Methanoplasma termitum, Candidatus Paceibacter, Eubacterium eligens, etc., but is not limited thereto.

In a case where Cpf1 protein is used as the endonuclease, the PAM sequence is 5′-TTN-3′ (N is A, T, C, or G) and the nucleotide sequence site to be cleaved (target site) may be a consecutive 17 bp- to 23 bp-long, for example, 21 bp- to 23 bp-long nucleotide sequence located adjacent to the 5′- and/or 3′-end of the 5′-TTN-3′ sequence in a target gene.

The endonuclease may be isolated from microbes or may be an artificial or non-naturally occurring enzyme as obtained by recombination or synthesis. For use, the endonuclease may be in the form of an mRNA pre-described or a protein pre-produced in vitro or may be included in a recombinant vector so as to be expressed in target cells or in vivo. In an embodiment, the endonuclease (e.g., Cas9, Cpf1, etc.) may be a recombinant protein made with a recombinant DNA (rDNA). The term “recombinant DNA” means a DNA molecule formed by artificial methods of genetic recombination, such as molecular cloning, to bring together homologous or heterologous genetic materials from multiple sources. For use in producing an endonuclease by expression in a suitable organism (in vivo or in vitro), recombinant DNA may have a nucleotide sequence that is reconstituted with optimal codons for expression in the organism which are selected from codons coding for a protein to be produced.

The endonuclease used herein may be a mutant target-specific nuclease in an altered form. The mutant target-specific nuclease may refer to a target-specific nuclease mutated to lack the endonuclease activity of cleaving double strand DNA and may be, for example, at least one selected from among mutant target-specific nucleases mutated to lack endonuclease activity but to retain nickase activity and mutant target-specific nucleases mutated to lack both endonuclease and nickase activities. As such, the mutation of the target-specific nuclease (e.g., amino acid substitution, etc.) may occur at least in the catalytically active domain of the nuclease (for example, RuvC catalyst domain for Cas9). In an embodiment, when the endonuclease is a Streptococcus pyogenes-derived Cas9 protein (SwissProt Accession number Q99ZW2(NP_269215.1); SEQ ID NO: 4), the mutation may be amino acid substitution at one or more positions selected from the group consisting of a catalytic aspartate residue (e.g., aspartic acid at position 10 (D10) for SEQ ID NO: 4, etc.), glutamic acid at position 762 (E762), histidine at position 840 (H840), asparagine at position 854 (N854), asparagine at position 863 (N863), and aspartic acid at position 986 (D986) on the sequence of SEQ ID NO: 4. A different amino acid to be substituted for the amino acid residues may be alanine, but is not limited thereto.

In another embodiment, the mutant target-specific nuclease may be a mutant that recognizes a PAM sequence different from that recognized by wild-type Cas9 protein. For example, the mutant target-specific nuclease may be a mutant in which at least one, for example, all of the three amino acid residues of aspartic acid at position 1135 (D1135), arginine at position 1335 (R1335), and threonine at position 1337 (T1337) of the Streptococcus pyogenes-derived Cas9 protein are substituted with different amino acids to recognize NGA (N is any residue selected from among A, T, G, and C) different from the PAM sequence (NGG) of wild-type Cas9.

In one embodiment, the mutant target-specific nuclease may have the amino acid sequence (SEQ ID NO: 4) of Streptococcus pyogenes-derived Cas9 protein on which amino acid substitution has been made for:
(1) D10, H840, or D10+H840;
(2) D1135, R1335, T1337, or D1135+R1335+T1337; or
(3) both of (1) and (2) residues.

As used herein, the term “a different amino acid” means an amino acid selected from among alanine, isoleucine, leucine, methionine, phenylalanine, proline, tryptophan, valine, asparagine, cysteine, glutamine, glycine, serine, threonine, tyrosine, aspartic acid, glutamic acid, arginine, histidine, lysine, and all variants thereof, exclusive of the amino acid retained at the original mutation positions in wild-type proteins. In one embodiment, “a different amino acid” may be alanine, valine, glutamine, or arginine.

As used herein, the term “guide RNA” refers to an RNA that includes a targeting sequence hybridizable with a specific base sequence (target sequence) of a target site in a target gene and functions to associate with a nuclease, such as Cas proteins, Cpf1, etc., and to guide the nuclease to a target gene (or target site) in vitro or in vivo (or in cells).

The guide RNA may be suitably selected depending on kinds of the nuclease to be complexed therewith and/or origin microorganisms thereof.

For example, the guide RNA may be at least one selected from the group consisting of:
CRISPR RNA (crRNA) including a region (targeting sequence) hybridizable with a target sequence;
trans-activating crRNA (tracrRNA) including a region interacting with a nuclease such as Cas protein, Cpf1, etc.; and
single guide RNA (sgRNA) in which main regions of crRNA and tracrRNA (e.g., a crRNA region including a targeting sequence and a tracrRNA region interacting with nuclease) are fused to each other.

In detail, the guide RNA may be a dual RNA including CRISPR RNA (crRNA) and trans-activating crRNA (tracrRNA) or a single guide RNA (sgRNA) including main regions of crRNA and tracrRNA.

The sgRNA may include a region (named “spacer region”, “target DNA recognition sequence”, “base pairing region”, etc.) having a complementary sequence (targeting sequence) to a target sequence in a target gene (target site), and a hairpin structure for binding to a Cas protein. In greater detail, the sgRNA may include a region having a complementary sequence (targeting sequence) to a target sequence in a target gene, a hairpin structure for binding to a Cas protein, and a terminator sequence. These moieties may exist sequentially in the direction from 5′ to 3′, but without limitations thereto. So long as it includes main regions of crRNA and tracrRNA and a complementary sequence to a target DNA, any guide RNA can be used in the present disclosure.

For editing a target gene, for example, the Cas9 protein requires two guide RNAs, that is, a CRISPR RNA (crRNA) having a nucleotide sequence hybridizable with a target site in the target gene and a trans-activating crRNA (tracrRNA) interacting with the Cas9 protein. In this context, the crRNA and the tracrRNA may be coupled to each other to form a crRNA:tracrRNA duplex or connected to each other via a linker so that the RNAs can be used in the form of a single guide RNA (sgRNA). In one embodiment, when a Streptococcus pyogenes-derived Cas9 protein is used, the sgRNA may form a hairpin structure (stem-loop structure) in which the entirety or a part of the crRNA having a hybridizable nucleotide sequence is connected to the entirety or a part of the tracrRNA including an interacting region with the Cas9 protein via a linker (responsible for the loop structure).

The guide RNA, specially, crRNA or sgRNA, includes a targeting sequence complementary to a target sequence in a target gene and may contain one or more, for example, 1-10, 1-5, or 1-3 additional nucleotides at an upstream region of crRNA or sgRNA, particularly at the 5′ end of sgRNA or the 5′ end of crRNA of dual RNA. The additional nucleotide(s) may be guanine(s) (G), but are not limited thereto.

In another embodiment, when the nuclease is Cpf1, the guide RNA may include crRNA and may be appropriately selected, depending on kinds of the Cpf1 protein to be complexed therewith and/or origin microorganisms thereof.

Concrete sequences of the guide RNA may be appropriately selected depending on kinds of the nuclease (Cas9 or Cpf1) (i.e., origin microorganisms thereof) and are an optional matter which could easily be understood by a person skilled in the art.

In an embodiment, when a Streptococcus pyogenes-derived Cas9 protein is used as a target-specific nuclease, crRNA may be represented by the following General Formula 1:

5′-(N_{cas9})_{I}-(GUUUUAGAGCUA)-(X_{cas9})ₘ-3′
(General Formula 1)

wherein:
N_{cas9} is a targeting sequence, that is, a region determined according to a sequence at a target site in a target gene (i.e., a sequence hybridizable with a sequence of a target site), I represents a number of nucleotides included in the targeting sequence and may be an integer of 15 to 30, 17 to 23 or 18 to 22, for example, 20;
the region including 12 consecutive nucleotides (GUUUUAGAGCUA; SEQ ID NO: 1) adjacent to the 3′-end of the targeting sequence is essential for crRNA;
X_{cas9} is a region including m nucleotides present at the 3′-terminal site of crRNA (that is, present adjacent to the 3′-end of the essential region); and
m may be an integer of 8 to 12, for example, 11 wherein the m nucleotides may be the same or different and are independently selected from the group consisting of A, U, C, and G.

In an embodiment, the X_{cas9} may include, but is not limited to, UGCUGUUUUG (SEQ ID NO: 2).

In addition, the tracrRNA may be represented by the following General Formula 2:

5[40 -(Y_{cas9})ₚ-(UAGCAAGUUAAAAUAAGGCUAGUCCGUUAUCAACUUGA
AAAAGUGGCACCGAGUCGGUGC)-3' (General Formula 2)

wherein,
the region represented by 60 nucleotides (UAGCAAGUUAAAAUAAGGCUAGUCCGUUAUCAACUUGAAAAAGUGGCACCG AGUCGGUGC) (SEQ ID NO: 3) is essential for tracrRNA,
Y_{cas9} is a region including p nucleotides present adjacent to the 3′-end of the essential region, and
p is an integer of 6 to 20, for example, 8 to 19 wherein the p nucleotides may be the same or different and are independently selected from the group consisting of A, U, C, and G.

Furthermore, sgRNA may form a hairpin structure (stem-loop structure) in which a crRNA moiety including the targeting sequence and the essential region of the crRNA and a tracrRNA moiety including the essential region (60 nucleotides) of the tracrRNA are connected to each other via an oligonucleotide linker (responsible for the loop structure). In greater detail, the sgRNA may have a hairpin structure in which a crRNA moiety including the targeting sequence and an essential region of crRNA is coupled with the tracrRNA moiety including the essential region of tracrRNA to form a double-strand RNA molecule with connection between the 3′ end of the crRNA moiety and the 5′ end of the tracrRNA moiety via an oligonucleotide linker.

In one embodiment, the sgRNA may be represented by the following General Formula 3:

5′-(N_{cas9})_{I}-(GUUUUAGAGCUA)-(oligonucleotide
linker)-(UAGCAAGUUAAAAUAAGGCUAGUCCGUUAUCAACUUGAAA
AAGUGGCACCGAGUCGGUGC)-3′ (General Formula 3)

wherein (N_{cas9})_{I} is a targeting sequence defined as in General Formula 1.

The oligonucleotide linker included in the sgRNA may be 3-5 nucleotides long, for example 4 nucleotides long in which the nucleotides may be the same or different and are independently selected from the group consisting of A, U, C, and G.

The crRNA or sgRNA may further contain 1 to 3 guanines (G) at the 5′ end thereof (that is, the 5′ end of the targeting sequence of crRNA).

The tracrRNA or sgRNA may further comprise a terminator inclusive of 5 to 7 uracil (U) residues at the 3′ end of the essential region (60 nt long) of tracrRNA.

The target sequence for the guide RNA may be about 17 to about 23 or about 18 to about 22, for example, 20 consecutive nucleotides adjacent to the 5′ end of PAM (Protospacer Adjacent Motif (for S. pyogenes Cas9, 5′-NGG-3′ (N is A, T, G, or C)) on a target DNA.

As used herein, the term “the targeting sequence” of guide RNA hybridizable with the target sequence for the guide RNA refers to a nucleotide sequence having a sequence complementarity of 50% or higher, 60% or higher, 70% or higher, 80% or higher, 90% or higher, 95% or higher, 99% or higher, or 100% to a nucleotide sequence of a complementary strand to a DNA strand on which the target sequence exists (i.e., a DNA strand having a PAM sequence (5′-NGG-3′ (N is A, T, G, or C))) and thus can complimentarily couple with a nucleotide sequence of the complementary strand.

In another embodiment, when the endonuclease is a Cpf1 system, the guide RNA (crRNA) may be represented by the following General Formula 4:

5′-n1-n2-A-U-n3-U-C-U-A-C-U-n4-n5-n6-n7-G-U-A-G-A-U-(Ncpf1)q-3′ (General Formula 4)

wherein,
n1 is null or represents U, A, or G,
n2 represents A or G,
n3 represents U, A, or C,
n4 is null or represents G, C, or A,
n5 represents A, U, C, or G, or is null,
n6 represents U, G, or C,
n7 represents U or G,
Ncpf1 is a targeting sequence including a nucleotide sequence hybridizable with a target site on a target gene and is determined depending on the target sequence of the target gene, and
q represents a number of nucleotides included therein and may be an integer of 15 to 30.

The target sequence (hybridizing with crRNA) of the target gene is a 15 to 30 (e.g., consecutive) nucleotide-long sequence adjacent to the 3′ end of PAM (5′-TTN-3′ or 5′-TTTN-3′; N is any nucleotide selected from A, T, G, and C.

In General Formula 4, the 5 nucleotides from the 6^{th} to the 10^{th} position from the 5′ end (5′ terminal stem region) and the 5 nucleotides from the 15^{th} (16^{th} when n4 is not null) to the 19^{th} (20^{th} when n4 is not null) position from the 5′ end are complementary to each other in the antiparallel manner to form a duplex (stem structure), with the concomitant formation of a loop structure composed of 3 to 5 nucleotides between the 5′ terminal stem region and the 3′ terminal stem region.

For the Cpf1 protein, the crRNA (e.g., represented by General Formula 4) may further comprise 1 to 3 guanine residues (G) at the 5′ end.

In crRNA sequences for Cpf1 proteins available from microbes of Cpf1 origin, 5′ terminal sequences (exclusive of targeting sequence regions) are illustratively listed in Table 1:

TABLE 1

| | 5′ Terminal Sequence (5′-3′) of |
|---|---|
| Microbe of Cpf1 origin | guide RNA (crRNA) |
| Parcubacteria bacterium | AAAUUUCUACU-UUUGUAGAU |
| GWC2011_GWC2_44_17 (PbCpf1) | |
| Peregrinibacteria bacterium | GGAUUUCUACU-UUUGUAGAU |
| GW2011_GWA_33_10 (PeCpf1) | |
| Acidaminococcus sp. BVBLG (AsCpf1) | UAAUUUCUACU-CUUGUAGAU |
| Porphyromonas macacae (PmCpf1) | UAAUUUCUACU-AUUGUAGAU |
| Lachnospiraceae bacterium ND2006 (LbCpi1) | GAAUUUCUACU-AUUGUAGAU |
| Porphyromonas crevioricanis (PcCpf1) | UAAUUUCUACU-AUUGUAGAU |
| Prevotella disiens (PdCpf1) | UAAUUUCUACU-UCGGUAGAU |
| Moraxella bovoculi 237 (MbCpf1) | AAAUUUCUACUGUUUGUAGAU |
| Leptospira inadai (LiCpf1) | GAAUUUCUACU-UUUGUAGAU |
| Lachnospiraceae bacterium MA2020 (Lb2Cpf1) | GAAUUUCUACU-AUUGUAGAU |
| Francisella novicida U112 (FnCpf1) | UAAUUUCUACU-GUUGUAGAU |
| Candidatus Methanoplasma termitum (CMtCpf1) | GAAUCUCUACUCUUUGUAGAU |
| Eubacterium eligens (EeCpf1) | UAAUUUCUACU--UUGUAGAU |

(-: denotes the absence of any nucleotide)

As used herein, the term “nucleotide sequence” hybridizable with a gene target site refers to a nucleotide sequence having a sequence complementarity of 50% or higher, 60% or higher, 70% or higher, 80% or higher, 90% or higher, 95% or higher, 99% or higher, or 100% to a nucleotide sequence (target sequence) of the gene target site (hereinafter used in the same meaning unless otherwise stated. The sequence homology can use a typical sequence comparison mean (e.g., BLAST)).

In the method, the transduction of the guide RNA and the RNA-guide endonuclease (e.g., Cas9 protein) into cells may be performed by directly introducing the guide RNA and the RNA-guide endonuclease into cells with the aid of a conventional technique (e.g., electroporation, etc.) or by introducing one vector (e.g., plasmid, viral vector, etc.) carrying both a guide RNA-encoding DNA molecule and a RNA-guide endonuclease-encoding gene (or a gene having a sequence homology of 80% or greater, 85% or greater, 90% or greater, 96% or greater, 97% or greater, 98% or greater, or 99% or greater thereto) or respective vectors carrying the DNA molecule or the gene into cells or through mRNA delivery.

In one embodiment, the vector may be a viral vector. The viral vector may be selected from the group consisting of negative-sense single-stranded viruses (e.g., influenza virus) such as retrovirus, adenovirus, parvovirus (e.g., adeno-associated virus (AAV)), corona virus, and orthomyxovirus; positive-sense single-stranded RNA viruses such as rhabdovirus (e.g., rabies virus and vesicular stomatitis virus), paramyxovirus (e.g., measles virus and sendai virus), alphavirus, and picornavirus; and double-stranded DNA viruses such as herpes virus (e.g., herpes simplex virus type 1 and 2, Epstein-Barr virus, cytomegalovirus), and adenovirus; poxvirus (e.g., vaccinia); fowlpox; and canarypox.

A vector carrying the Cas9 protein, the guide RNA, a ribonucleoprotein containing both of them, or at least one thereof may be delivered into a body or cells, using a suitable one of well-known techniques such as electroporation, lipofection, viral vector, nanoparticles, and PTD (protein translocation domain) fusion protein. The Cas9 protein and/or guide RNA may further include a pertinent nuclear localization signal (NLS) for the intranuclear translocation of the Cas9 protein, the guide RNA, or the ribonucleoprotein containing both of them.

As used herein, the term “cleavage” in a target site means the breakage of the covalent backbone in a polynucleotide. The cleavage includes enzymatic or chemical hydrolysis of a phosphodiester bond, but is not limited thereto, and may be performed by various other methods. Cleavage may be possible on both single strands and double strands. The cleavage of a double-strand may result from the cleavage of the two distinct single strands, with the consequent production of blunt ends or staggered ends.

### Advantageous Effect

The formation and regeneration of blood vessels in the myocardial infarction or lower limb ischemia model of the present disclosure is essential, but there has been an urgent need for developing a method for promoting the formation and regeneration of blood vessels. Therefore, the Ang-1- and VEGF-secreting stem cell of the present disclosure helps the regeneration of blood vessels in a patient suffering from a cardiovascular disease such as myocardial infarction, lower limb ischemia, and so forth and thus can be advantageously used for the prevention and treatment thereof.

### BRIEF DESCRIPTION OF THE DRAWINGS

FIG. 1 shows a schematic diagram of a vector structure for use in generating Ang-1-secreting umbilical cord mesenchymal stem cells, and the secretion of Ang-1 from the cells generated therewith as measured by western blotting and ELISA assays.

FIG. 2 shows a schematic diagram of a vector structure for use in VEGF-secreting umbilical cord mesenchymal stem cells, and the secretion of VEGF from the cells generated therewith as measured by western blotting and ELISA assays.

FIGS. 3a and 3b are views illustrating the increase of indel efficiency by CRISPR/Cas9 RNP in Jurkat cells, wherein the CRISPR/Cas9 RNP is prepared to deliver a vector for generating Ang-1- or VEGF-secreting cells.

FIG. 4 shows photographic images illustrating lower limb injury in the mouse lower limb ischemia models to which Ang-1-secreting umbilical cord mesenchymal stem cells or VEGF-secreting umbilical cord stem cells were injected.

FIGS. 5a to 5c shows the effects of Ang-1- and VEGF-secreting umbilical cord mesenchymal stem cells in terms of the viability of cardiomyocytes (proliferation assay) (5a), the degree of vascular formation (5b), and the expression levels of main factors (5c).

FIG. 6 shows degrees of fibrosis in the heart tissues of the myocardial infarction models treated with Ang-1-secreting umbilical cord mesenchymal stem cells and VEGF-secreting umbilical cord mesenchymal stem cells alone or in combination in terms of scar area (% of LV (left ventricular) area), infarcted wall thickness (mm), and LV expansion index.

FIG. 7a shows in vivo CINE-f-MRI images accounting for ejection fractions of the rat hearts in myocardial infarction models co-treated with Ang-1-MSC and VEGF-MSC and FIG. 7b is a graph showing infarction sizes in the models.

FIG. 8 shows fluorescence images illustrating degrees of vascular formation in myocardial infarction models treated with either or both of Ang-1-secreting umbilical cord mesenchymal stem cells and VEGF-secreting umbilical cord mesenchymal stem cells.

### MODE FOR CARRYING OUT THE INVENTION

Hereinafter, the present disclosure will be described in more detail with reference to Examples, which are merely illustrative and are not intended to limit the scope of the present disclosure. It is apparent to those skilled in the art that the Examples described below may be modified without departing from the essential gist of the disclosure.

### Example 1: Generation of Ang-1- and VEGF-Secreting Cell by Using CRISPR/Cas9 RNP

1.1. Generation of Ang-1-Secreting Cell

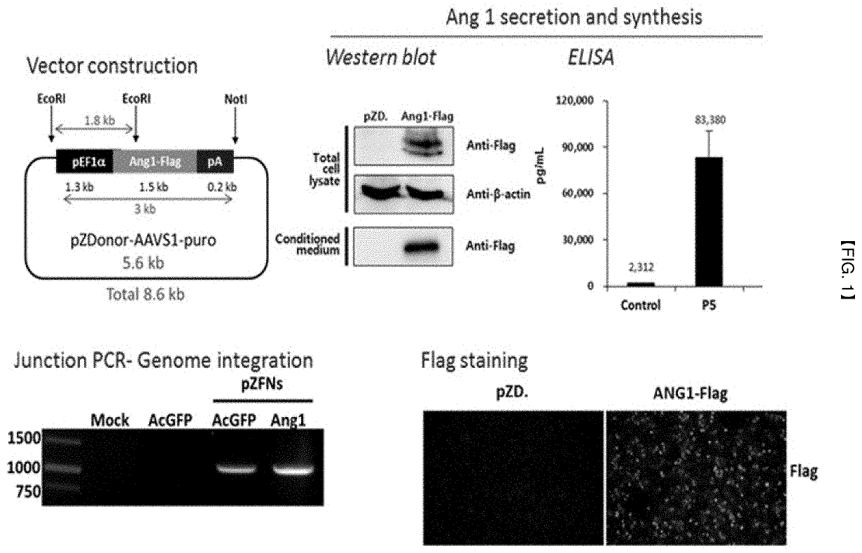

An Ang-1 gene (GenBank Accession No. NM_001146.4) was inserted into a pZDonor vector (Sigma Aldrich) to construct a recombinant vector for Ang-1 expression (see FIG. 1). In addition, AAVS1-targeting CRISPR/Cas9 RNP (ToolGen, Inc) was prepared (Cas9: Streptococcus pyogenes-derived Cas9 protein; the targeting sequence of sgRNA for AAVS1: gucaccaauccugucccuag; refers to General Formula 3 supra, with respect to the entire sequence).

The AAVS1-targeting CRISPR/Cas9 RNP and the pZDonor carrying the Ang-1 gene were co-transfected into umbilical cord mesenchymal stem cells. The umbilical cord mesenchymal stem cells were prepared as follows: human umbilical cord was treated and centrifuged. After removal of the supernatant, the cells were placed in a T25 flask and cultured in a 37° C. incubator provided with 5% CO₂. After 7 days, cells adherent to the flask were subjected to chromosomal assay while the non-adhering umbilical cord cells were transferred into a T25 flask containing a modified minimum essential medium supplemented with 20% fetal bovine serum (FBS) and 4 ng/mL basic fibroblast growth factor. After 5-7 days of culturing, whether the cells adhered to the bottom and were growing was identified. When the cells stably proliferated, the medium was changed. Then, the cells were cultured to 80% confluency, with the exchange of the medium with a fresh one twice per week.

The CRISPR/Cas9 RNP cleaves an AAVS site on cell genomic genes to insert a desired gene (e.g., Ang-1 gene) into the cleaved site, thereby generating Ang-1-secreting umbilical cord mesenchymal stem cells (Ang-1-MSC). The Ang-1 secretion of the generated Ang-1-MSC was assayed by western blotting, ELISA, PCR, and fluorescent immunostaining (Flag), and the results are depicted in FIG. 1.

1.2. Generation of VEGF-Secreting Cel

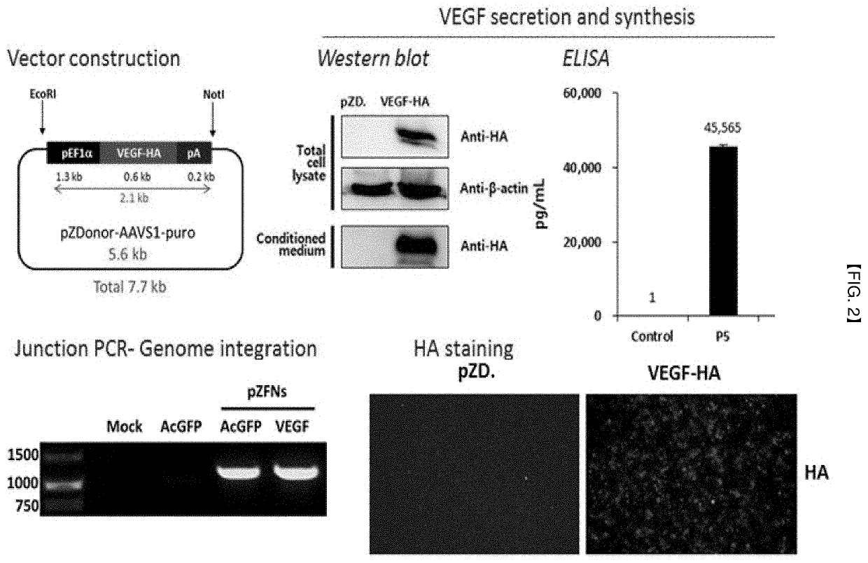

A VEGF gene (GenBank Accession No. NM_001171623.1) was inserted into a pZDonor vector (Sigma-Aldrich) to construct a recombinant vector for VEGF expression (FIG. 2). In addition, AAVS1-targeting CRISPR/Cas9 RNP (ToolGene Inc.) was prepared (Cas9: Streptococcus pyogenes-derived Cas9 protein; the targeting sequence of sgRNA for AAVS1: gucaccaauccugucccuag; refers to General Formula 3 supra, with respect to the entire sequence).

The above-prepared AAVS1-targeting CRISPR/Cas9 RNP and the pZDonor carrying the VEGF gene were co-transfected into human umbilical cord mesenchymal stem cells (see Example 1.1).

The CRISPR/Cas9 RNP cleaves an AAVS site on cell genomic genes to insert a desired gene (e.g., VEGF gene) into the cleaved site, thereby generating VEGF-secreting umbilical cord mesenchymal stem cells (VEGF-MSC). The VEGF secretion of the generated VEGF-MSC was assayed by western blotting, ELISA, PCR, and fluorescent immunostaining (Flag), and the results are depicted in FIG. 2.

The assays were conducted as follows:

RT-PCR Analysis

After RNA isolation using Trizol, cDNA was synthesized using an olig-dT primer and a reverse transcriptase. cDNA synthesis started with reverse transcription at 42°_ C. for one hour, followed by thermal treatment at 95° C. for 10 min to stop the enzymatic activity. Primers for a gene of interest were designed and used for PCR (primers: Fwd: 5′-cggaactctgccctctaacg-3′; Rev: 5′-tgaggaagagttcttgcagct-3′).

Western Blot

The protein concentration in an isolated protein solution was measured by BCA assay and a predetermined amount of the protein solution was run on a 10% SDS-PAGE gel by electrophoresis before transfer onto a PVDF membrane. This membrane was incubated with a primary antibody (Sigma Aldrich) at 4° C. for 12 hours and then washed to remove the unbound antibody. Subsequently, incubation with an HRP-conjugated secondary antibody (Vector Laboratories) was done at room temperature for one hour. After completion of the reaction, protein expression was analyzed with ECL (Amersham).

Immunocytochemistry-Fluorescent Staining

Fixed cells were reacted with a primary antibody at 4° C. for 12 hours and washed, followed by incubation with fluorescein-conjugated goat anti-rabbit IgG at room temperature for one hour. The cells thus stained were mounted on a glass slide and observed under a Zeiss confocal microscope.

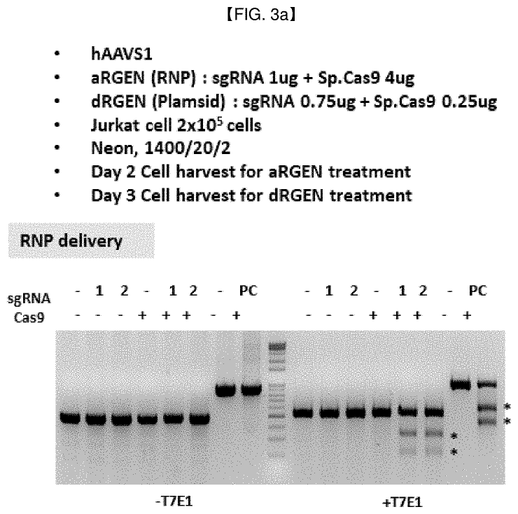

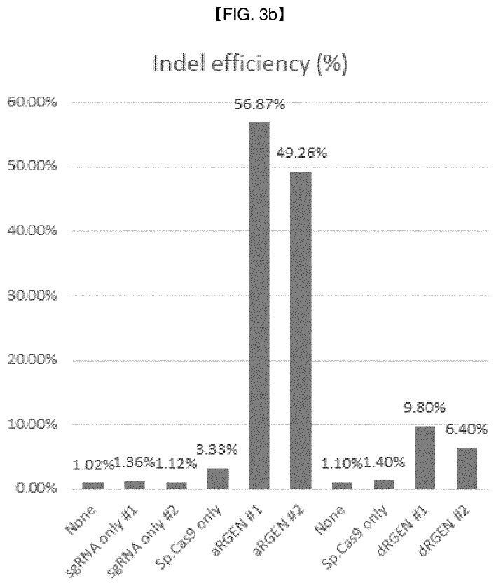

In addition, gene editing (Indel: insertion and/or deletion) efficiency of the above prepared CRISPR/Cas9 RNP was tested in Jurkat cells (ATCC) and the results are depicted in FIGS. 3a and 3b.

(In FIGS. 3 and 3b,
none: mock transfection;
sgRNA #1: transfected with 5′-GTCACCAATCCTGTCCCTAG(TGG)-3′ (hAAVS1 #1; PAM sequence in the parentheses)-targeting guide RNA (sgRNA) alone;
sgRNA #2: transfected with 5′-ACCCCACAGTGGGGCCACTA(GGG)-3′ (hAAVS1 #2; PAM sequence in the parentheses)-targeting sgRNA alone;
Sp.cas9 only: transfected with cas9 protein alone;
aRGEN1: transfected with hAAVS1 #1-targeting sgRNA #1 plus cas9;
aRGEN2: transfected with hAAVS1 #2-targeting sgRNA #2 plus cas9;
dRGEN1: transfected with hAAVS1 #1-targeting sgRNA #1 plus cas9-carrying plasmid; and
dRGEN2: transfected with hAAVS1 #2-targeting sgRNA #2 plus cas9-carrying plasmid)

As shown in FIGS. 3a and 3b, the RNP form was observed to have higher efficiency in intracellular delivery and gene editing than plasmid form.

### Example 2: Protective Effect on Cardiomyocyte

2.1. Human Cardiomyocyte Culturing

Cardiomyocytes were suspended in DMEM (culture medium) containing 5% (v/v) FBS, 5% (v/v) HS (horse serum), 20 μg/ml gentamicin and 2.5 μg/ml amphotericin B, plated at a density of 1×10⁶ cells/ml (10 ml) into 10-cm culture dishes, and maintained at 37° C. in a 5% CO₂/95% atmosphere in an incubator. After 2-3 weeks of in vitro culture, the cells were treated with AGE-albumin and used in analyzing apoptosis-related properties.

2.2. Cell Viability (MTT Assay)

Human cardiomyocytes prepared in Example 2.1 were seeded at a density of 2×10³ cells/well into 96-well plates. When reaching 80% confluence, the human cardiomyocytes were treated with 50 nM AGE-albumin for 24 hours and then with Ang-1-MSC (Ang-1-secreting umbilical cord mesenchymal stem cells) or VEGF-MSC (VEGF-secreting umbilical cord mesenchymal stem cells) (see Example 1) for 24 hours. Thereafter, the cells were rinsed with PBS and examined for viability using an MTT [3-(4,5-dimethylthiazol-2-yl)-2,5-diphenyl tetrazolium bromide] assay. Living cells reduce the yellow MTT compound into purple formazan, which is soluble in dimethyl sulfoxide (Me₂SO). In each well, the cells were incubated for 2 hours with the MTT compound at 0.5 mg/ml and then added with DMSO (Sigma-Aldrich). The intensity of blue staining in the culture medium was measured at 540 nm using a spectrophotometer and was expressed as proportional amounts of living cells.

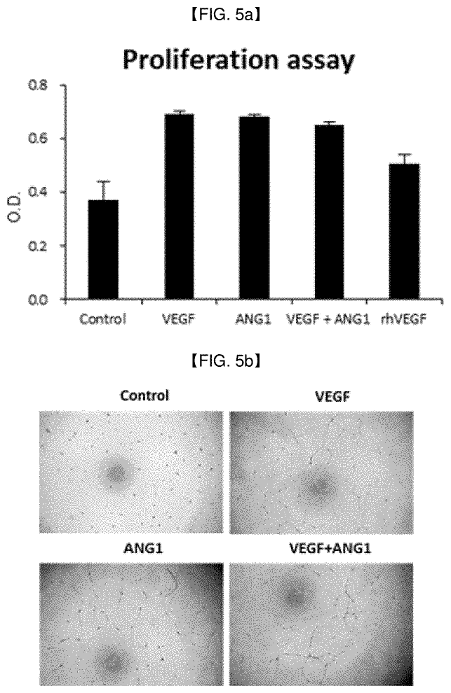

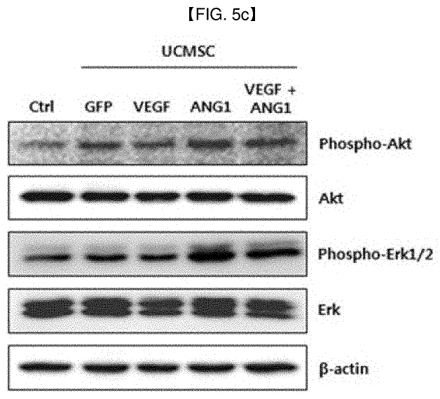

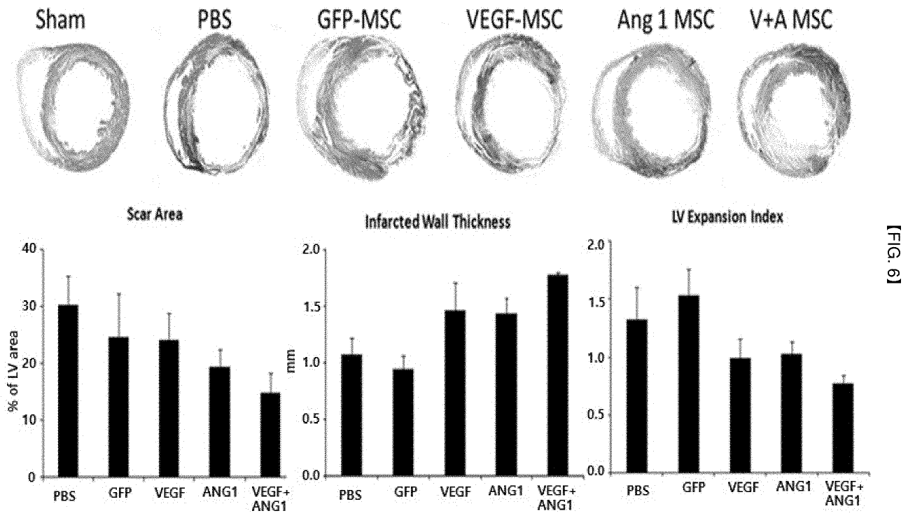

The results are shown in FIGS. 5a (proliferation assay result; cell viability) and 5b (views of stereoscopic optical microscope; angiogenesis rate) (GFP: GFP-MSC, VEGF: VEGF-MSC, ANG1: ANG1-MSC, VEGF+ANG1: mixture of VEGF-MSC and ANG1-MSC, and rhVEGF: recombinant human VEGF (RND system)).

As shown in FIGS. 5a and 5b, human cardiomyocytes, when treated with AGE-albumin, underwent cell death and thus decreased in cell viability. In contrast, treatment with ANG-1- and/or VEGF-secreting umbilical cord mesenchymal stem cells increased cell viability and angiogenesis rate in primary human cardiomyocyte. In addition, a remarkably higher effect was brought about in angiogenesis rate by VEGF-MSC than the recombinant protein rhVEGF, which is attributed to the fact that VEGF-MSC contributes to secrete VEGF.

These data indicate that ANG-1- or VEGF-secreting umbilical cord mesenchymal stem cells have a protective effect on cardiac muscle cell death (inhibitory effect on cardiomyocyte death) at higher efficiency than protein forms of ANG-1 or VEGF.

2.3. Measurement of Angiogenic and Vasculogenic Factor (Western Blotting)

The cardiomyocytes treated with each of the stem cells in Example 2.2 were powdered with liquid nitrogen and lysed in RIPA buffer (Abcam). After centrifugation, the supernatant was taken as a solution of proteins from the stem cell-treated cardiomyocytes. The protein concentration in an isolated protein solution was measured using BCA (Life technologies) according to the manufacturer's instructions and a predetermined amount of the protein solution (total protein amount: 30 μg) was run on a 10% SDS-PAGE gel by electrophoresis before transfer onto a PVDF membrane. This membrane was incubated with a primary antibody (Sigma Aldrich) at 4° C. for 12 hours and then washed to remove the unbound antibody. Subsequently, incubation with an HRP-conjugated secondary antibody (Vector Laboratories) was done at room temperature for one hour. After completion of the reaction, protein expression was analyzed with ECL (Amersham). The results are given in FIG. 5c. As shown in FIG. 5c, the most prominent increase in the expression of Akt and p-ERK1/2, which are essential for angiogenesis and vasculogenesis was observed upon treatment with ANG-1- and/or VEGF-secreting umbilical cord mesenchymal stem cells.

### Example 3: Protective Effect of Ang-1-MSC or VEGF-MSC on Cardiac Muscle Cell Death in Myocardial Infarction Model (In Vivo Assay)

3.1. Establishment of Myocardial Infarction Animal Model

Sprague-Dawley rats, each weighing 250-300 g, were prepared, and anaesthetized with a combination of ketamine (50 mg/kg) and xylazine (4 mg/kg). A 16-gauge catheter was inserted into the bronchus and connected with an artificial respirator. After the animal was fixed with a tape against a flat plate to secure the limbs and the tail, a 1-1.5 cm vertical incision was made left from the sternum, and the pectoralis major muscle was separated from the pectoralis minor muscle to ascertain the space between the 5^{th} and 6^{th} ribs. Then, the muscle therebetween was carefully incised at 1 cm in a widthwise direction. A retractor was pushed in between the 5^{th} and 6^{th} ribs which were then separated further from each other. Since the upper part of the heart is typically covered with the thymus in rats, the thymus was pulled to the head using an angle hook to clearly view the heart. The figure of the left coronary artery was scrutinized to determine the range of artery branches to be tied. The LAD (left anterior descending artery) located 2-3 mm below the junction of the pulmonary conus and the left atrial appendage was ligated with 6-0 silk. Subsequently, the 5^{th} and 6^{th} ribs were positioned to their original places, and the incised muscle was sutured with MAXON 4-0 filament, followed by withdrawing air from the thoracic cavity through a 23-gauge needle syringe to spread the lungs fully. The skin was sutured with MAXON 4-0 filament. The catheter was withdrawn, and viscous materials were removed from the pharynx. After operation, a pain-relieving agent (Buprenorphine 0.025 mg/kg) was subcutaneously injected every 12 hours.

3.2. Protective Effect of Ang-1-MSC or VEGF-MSC

To the myocardial infarction animal model prepared above, the Ang-1-secreting umbilical cord mesenchymal stem cells (Ang-1-MSC) and/or VEGF-secreting umbilical cord mesenchymal stem cells (VEGF-MSC) were injected (injection dose: a total of 30 μl, 1×10⁶ cells in 30 μl). The cardiomyocytes were stained with cresyl violet and counted under a microscope.

The results are given in FIG. 6. FIG. 6 shows images of stained heart tissues (upper panels) and graphs pertaining to infarction (lower panels). Depicted in the graphs are quantitated scar areas (% of LV (left ventricular) area), with lower numerical values accounting for lower levels of fibrosis in the heart (left), infarcted wall thicknesses (mm), with higher numerical values accounting for better rehabilitation from myocardial infarction (middle), and left ventricular (LV) expansion indices, with lower numerical values accounting for better rehabilitation from myocardial infarction. As shown in FIG. 6, the injection of the Ang-1- and/or VEGF-secreting mesenchymal stem cells reduced fibrosis areas (blue) and myocardial infarction areas (red) in the heart cells of the rats before or after myocardial infarction, with the observation of increasing therapeutic effects on myocardial infarction in the following order MSC<ANG1-MSC<VEGF-MSC<ANG1-MSC+VEGF-MSC (A+V MSC).

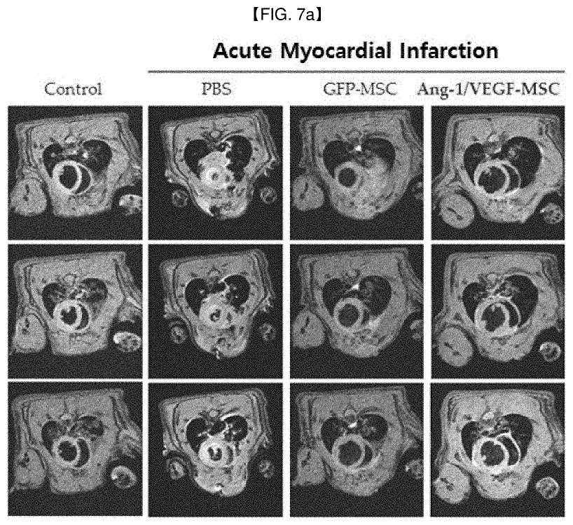

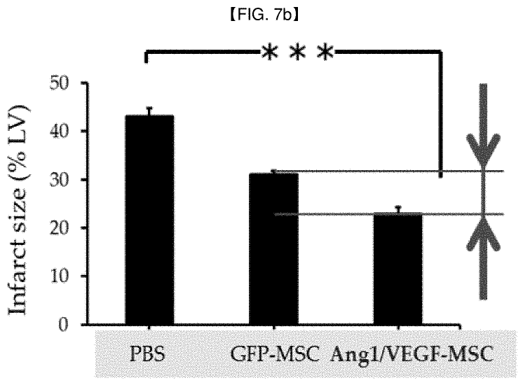

In addition, ejection fractions of the rat heart in the myocardial infarction models co-treated with Ang-1-MSC and VEGF-MSC are shown in FIG. 7a, as imaged in vivo by the CINE-f-MRI while infarction sizes are graphed in FIG. 7b. As can be seen in FIGS. 7a and 7b, the ejection fraction of the co-treated heart was prominently increased, compared to that of general MSC-treated heart.

### Example 4: Protective Effect of Ang-1-MSC or VEGF-MSC on Cardiac Muscle Cell Death in Lower Limb Ischemia Model (In Vivo Assay)

4.1. Establishment of Rat Lower Limb Ischemia Model

As experimental animals, male Balb/c-nu mice were used. Animal model establishment was conducted in a clean and sterile environment under the anesthesia by N20:02=1:1 (v:v), isoflurane inhalation.

After anesthesia, incision of about 2 cm was made on the skin. Then, 3-0 surgical silk was applied to an accurate site (5-6 mm below iliac arteries or superficial femoral arteries and inguinal ligament) for ligation, followed by closing the skin with a skin clip.

4.2. Protective Effect on Lower Limb Muscle Cell Death

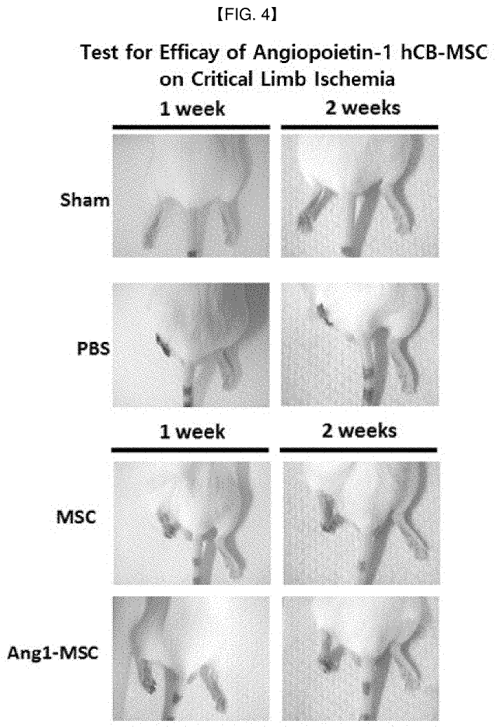

To examine the protective effect of Ang-1-MSC or VEGF-MSC on lower limb muscle cell death in lower limb ischemia model, a total of 10⁶ cells of Ang-1-MSC was injected into the tissue of the rat lower limb ischemia model established above. After one and two weeks, the lower limbs of the mice were observed and are shown in FIG. 4.

FIG. 4 shows photographic images of lower limbs of the mouse lower limb ischemia models to which Ang-1-MSC, MSC (positive control), and PBS (negative control) were injected (Sham: normal mouse with no lower limb ischemia induced therein). As shown in FIG. 4, lower limb muscle cell death was reduced in the Ang-1-MSC-injected mouse, compared to the MSC- or PBS-injected mouse.

### Example 5: Immunohistochemistry (IHC)

Immunohistochemistry was conducted on heart tissues from normal or myocardial infarction rats. Normal or myocardial infarction heart tissues were fixed with 4% paraformaldehyde in a 0.1 M neutral phosphate buffer, cryopreserved overnight in a 30% sucrose solution, and then sectioned on a cryostat (Leica CM 1900) at a 10 μm thickness. Paraffin-embedded tissues were cut into 10 μm-thick sections, deparaffinized with xylene, and rehydrated with a series of graded ethanol. Normal goat serum (10%) was used to block non-specific protein binding. The tissue sections were incubated overnight at 4° C. with the following primary antibodies: rabbit anti-alpha-SMA antibody (Abcam), mouse anti-human albumin antibody (1:200, R&D System), and goat anti-Iba1 antibody (1:500, Abcam). Then, the tissue sections were washed three times with PBS before incubation for 1 hour at room temperature with Alexa Fluor 633 anti-mouse IgG (1:500, Invitrogen). After washing the secondary antibodies three times with PBS, coverslips were mounted onto glass slides using the Vectashield mounting medium (Vector Laboratories), and observed under a laser confocal fluorescence microscope (LSM-710, Carl Zeiss).

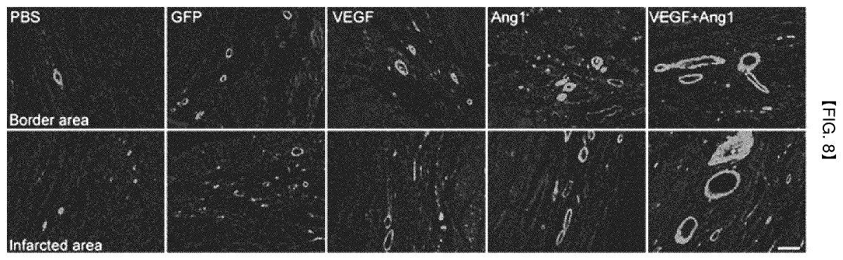

The results are depicted in FIG. 8. As shown in FIG. 8, the alpha-SMA factor, which is responsible for angiogenesis, was most intensively stained upon treatment with either or both of Ang-1-MSC and VEGF-MSC in the rat heart tissues.

1-6. (canceled)

7. A method of vascular formation, comprising administering at least one mesenchymal stem cell selected from the group consisting of a VEGF-secreting mesenchymal stem cell and an Ang-1-secreting mesenchymal stem cell, or a culture of the mesenchymal stem cell, to a subject in need of vascular formation,
wherein the VEGF-secreting mesenchymal stem cell contains a VEGF gene inserted thereinto and secrets VEGF, and the Ang-1-secreting mesenchymal stem cell contains an Ang-1 gene inserted thereinto and secrets Ang-1.

8. The method of claim 7, wherein the mesenchymal stem cell is an umbilical cord-derived mesenchymal stem cell.

9. The method of claim 7, wherein the VEGF gene and the Ang-1 gene are inserted into a safe harbor site in the genome of the mesenchymal stem cell.

10. A method of inhibiting ischemic cell death, comprising administering at least one mesenchymal stem cell selected from the group consisting of a VEGF-secreting mesenchymal stem cell and an Ang-1-secreting mesenchymal stem cell, or a culture of the mesenchymal stem cells, to a subject in need of inhibiting ischemic cell death,
wherein the VEGF-secreting mesenchymal stem cell contains a VEGF gene inserted thereinto and secrets VEGF, and the Ang-1-secreting mesenchymal stem cell contains an Ang-1 gene inserted thereinto and secrets Ang-1.

11. The method of claim 10, wherein the mesenchymal stem cell is an umbilical cord-derived mesenchymal stem cell.

12. The method of claim 10, wherein the VEGF gene and the Ang-1 gene are inserted into a safe harbor site in the genome of the mesenchymal stem cell.

13. A method of preventing or treating a cardiovascular disease, comprising administering at least one mesenchymal stem cell selected from the group consisting of a VEGF-secreting mesenchymal stem cell and an Ang-1-secreting mesenchymal stem cell, or a culture of the mesenchymal stem cells, to a subject in need of preventing or treating a cardiovascular disease,
wherein the VEGF-secreting mesenchymal stem cell contains a VEGF gene inserted thereinto and secrets VEGF, and the Ang-1-secreting mesenchymal stem cell contains an Ang-1 gene inserted thereinto and secrets Ang-1.

14. The method of claim 13, wherein the cardiovascular disease is stroke, myocardial infarction, angina pectoris, lower limb ischemia, hypertension, or arrhythmia.

15. The method of claim 13, wherein the mesenchymal stem cell is an umbilical cord-derived mesenchymal stem cell.

16. The method of claim 13, wherein the VEGF gene and the Ang-1 gene are inserted into a safe harbor site in the genome of the mesenchymal stem cell.
